(19) United States
(12) Patent Application Publication
(10) Pub. No.: US 2014/0080846 A1
(43) Pub. Date: Mar. 20, 2014

(54) Method of treating orphan respiratry diseases using doxofylline

(71) Applicants: William Wayne Howard, Morristown, NJ (US); Russell Francis Somma, Sparta, NJ (US)

(72) Inventors: William Wayne Howard, Morristown, NJ (US); Russell Francis Somma, Sparta, NJ (US)

(21) Appl. No.: 13/573,435

(22) Filed: Sep. 14, 2012

(51) Int. Cl. A61K 31/52; A61P 11/00

(52) U.S. Cl. 514/263.23

(57) ABSTRACT

A method of treating orphan respiratory disease.

### BACKGROUND OF THE INVENTION

1. Field of the Invention

The present invention relates to the use of doxofylline to treat respiratory disorders that are uncommon in the general population and that are often referred to as Orphan Diseases.

2. Description of the Related Art

In the United States, Orphan Diseases are defined as ones that affect less than 200,000 patients. In regard to respiratory diseases, the following are Orphan Respiratory Diseases of interest to the present invention.
Cystic Fibrosis
Bronchiolitis Obliterans
Bronchiectasis
Emphysema/COPD secondary to antitrypsin deficiency
Reactive Airway Dysfunction Syndrome (RADS) in 9/11 First Responders and Survivors
World Trade Center Cough in 9/11 First Responders and Survivors
Other orphan respiratory diseases that cause impaired lung function due to obstruction of the lung's airway passages

Cystic fibrosis (CF) is associated with a defective gene that causes the body to produce abnormally thick and sticky mucus. This mucus builds up in the bronchial passages of the lungs and in the body of the pancreas. This build-up of mucus results in life-threatening lung infections and serious digestion problems. Cystic fibrosis is a recessive genetic disorder affecting primarily the lungs, and also the pancreas, liver, and intestine. It is characterized by abnormal transport of chloride and sodium across an epithelium, leading to thick, viscous secretions (Yankaskas et al, 2004).

CF is caused by a mutation in the gene for the protein cystic fibrosis transmembrane conductance regulator (CFTR). This protein is required to regulate the components of sweat, digestive juices, and mucus. CFTR regulates the movement of chloride and sodium ions across epithelial membranes, such as the alveolar epithelia located in the lungs. Although most people without CF have two working copies of the CFTR gene, only one is needed to prevent cystic fibrosis due to the disorder's recessive nature. CF develops when neither gene works normally and therefore has autosomal recessive inheritance (Mitchell et al, 2007).

The name cystic fibrosis refers to the characteristic scarring (fibrosis) and cyst formation within the pancreas, first described by Anderson in 1938. Significantly impaired breathing is the most serious symptom of CF. Impaired breathing is a result of frequent lung infections associated with CF. These respiratory infections are treated with antibiotics and other medications. Other symptoms, including sinus infections, poor growth, and infertility affect other parts of the body (Harden, 2004; O'Malley, 2009; Makker, 2009).

In addition to treatment with antibiotics, bronchodilators and anti-inflammatory drugs have also been employed to treat CF (Colombo, 2003; Balfour-Lynn, 2009). Theophylline is a member of the xanthine class of drugs and is known to have both bronchodilator and anti-inflammatory effects (Sullivan et al, 1994). Further, intravenous theophylline has been tested in CF patients and positive results were reported on Forced Expiratory Volume at 1 second (FEV1) and Forced Expiratory Flow at 0.75 seconds (FEF 0.75) (Pan et al, 1989). However, as noted by Colombo, theophylline has received limited use in the treatment of CF due to its high side effect profile and the need for serum monitoring of theophylline. Serum monitoring is needed because theophylline is known as a “narrow therapeutic index” drug i.e. there is a small difference between a therapeutic amount and an amount which causes potentially serious side effects.

Bronchiolitis Obliterans is a lung disease that results in scarring of the small airways in the lung. Bronchiolitis Obliterans, also called Obliterative bronchiolitis and constrictive bronchiolitis, is a rare and life-threatening form of non-reversible obstructive lung disease in which the bronchioles (small airway branches) are compressed and narrowed by fibrosis (scar tissue) and/or inflammation. The scarring will result in less air exchange in the airways and over time, the airways may be blocked. (Epler, 2010). Bronchiolitis Obliterans refers to the fact that the inflammation or fibrosis of the bronchioles partially or completely obliterates the airways. (Colby, 1998).

Bronchiectasis has been defined as an abnormal and permanent dilatation of the cartilage-containing airways (bronchi) (Feldman, 2011). The primary feature of bronchiectasis is the presence of airway inflammation, along with bacterial infection, specifically non-clearing infection. Bronchiectasis treatment centers on air way clearance but anti-inflammatories have been used. Therapy with inhaled corticosteroids and oral macrolides may reduce airway inflammation in patients with bronchiectasis (O'Donnell, 2008). Tsang et al demonstrated that therapy with inhaled fluticasone lead to clinical improvement in patients who had been treated with 500 ug of inhaled fluticasone twice per day compared to placebo (Tsang et al 2005). The role of anti-inflammatories and bronchodilators in treatment of bronchiectasis has been evaluated by Martinez-Garcia et al, (2011) and positive results were obtained from a combination of a steroid (budesonide) and a LABA (formoterol) in an inhaled dosage form (Martinez-Garcia et al, 2011).

Alpha 1-antitrypsin deficiency (al-antitrypsin deficiency, A1AD or antitrypsin deficiency) is a genetic disorder that causes defective production of alpha 1-antitrypsin (A1AT). This causes a decrease in A1AT activity in the blood and lungs. It can also affect the liver (Stoller et al, 2005). There are several forms and degrees of deficiency, principally depending on whether the sufferer has one or two copies of the affected gene because it is a co-dominant trait. Severe A1AT deficiency causes emphysema or COPD in adult life in many people with the condition, especially if they are exposed to cigarette smoke (Needham et al, 2004). The treatment of emphysema or COPD associated with antitrypsin deficiency follows the standards of care for these diseases, meaning, use of bronchodilators such as LABAs and anti-inflammatories such as inhaled steroids (Kaplan, 2010; Corda et al, 2008; Stoller et al, 2005).

Reactive Airways Dysfunction Syndrome or RADS is a term proposed by Brooks et al (1985) to describe an asthma-like syndrome developing after a single exposure to high levels of an irritating vapor, fume, or smoke. It involves coughing, wheezing, and dyspnea. More recent work in regard to the attack on the World Trade Center in 2001 has expanded the definition of RADS to include more than one exposure in an acute or chronic setting (Prezant et al, 2008). It can also manifest in adults with exposure to high levels of chlorine, ammonia, acetic acid or sulfur dioxide, creating symptoms like asthma (Brooks et al, 1985).

The net effect of such brief exposure can be long term loss of lung function. Prezant et al observed that six years after the WTC attack, firemen who showed initial loss of lung function have persistent loss six years later in measures such as FEV1 (Prezant et al, 2008). Not only was there no indication of recovery but the trend was for continued loss, especially among smokers. Other analyses conducted on the data from the 9/11 attack shows that first responders suffer predominantly from obstructive lung disorders (Weiden et al, 2010).

Treatment for Chronic Obstructive Lung Disease (COPD) is currently based on long acting beta agonists (LABAs) and inhaled cortico-steroids (ICS) (Wedzicha et al, 2012). Importantly, ICS has been shown to improve lung function of firefighters exposed to the dust cloud at the World Trade Center following the attack and whose lung function had declined measurably after that event (Banauch et al, 2008).

While each disease has its own well defined characteristics, all of them may be treatable with the combination of a bronchodilator and an anti-inflammatory agent. Long acting beta agonists (LABA) can be combined with a steroid to create both bronchodilation and anti-inflammatory effects. Representative references for this observation include the following. For Cystic Fibrosis see Colombo, 2003 and Balfour-Lynn, 2009; for bronchiectasis see Martinez-Garcia et al, 2011; for bronchiolitis see Bergeron et al, 2007; for emphysema or COPD secondary to antitrypsin deficiency see Kaplan, 2010; for World Trade Center Cough see Friedman et al, 2006; for reactive airways dysfunction syndrome see Weiden et al, 2010 who note that RADS is primarily an obstructive lung disease and Wedzicha et al, 2012 for treatment of obstructive lung diseases. Wedzicha et al note that long acting beta agonists (LABA) or the anti-cholinergic drug are often co-administered with an inhaled cortico-steroid so the steroid is utilized as an anti-inflammatory agent.

As noted, a common combination is either a long acting beta agonist or anti-cholinergic combined with a steroid e.g. Advair® (fluticasone and salmeterol) and Symbicort® (budesonide/formoterol fumarate dihydrate). In 2010, Advair was the fourth largest selling pharmaceutical product in the United States with total sales of $3.7 billion (Drug Topics, June, 2011). Furthermore, the dominant route of administration of these drug combinations is inhalation (Lareau and Yawn, 2010). A significant down side to this approach is that inhaled drugs suffer broadly from non-compliance across a wide range of respiratory conditions.

Non-Compliance in Asthma

Although not an Orphan Disease, asthma is an obstructive respiratory disease and compliance to asthma treatments is likely indicative of compliance in Orphan Diseases such as Cystic Fibrosis. Compliance with inhaled medication in patients with asthma is known to be poor, with about half of patients under-medicating with respect to current guidelines for optimum disease control. Chatkin et al. (2006) conducted a study in 131 asthma patients in Brazil and found that the overall rate of compliance was only 51.9%. Lacasse et al. (2005) performed a similar study in Canada and concluded that patients took between 48% and 96% of their prescriptions. Finally, Bender et al. (2000) compared four adherence assessment methods: child report, mother report, canister weight, and electronic measurements of metered dose inhaler (MDI) actuation. Participants included 27 children with mild-to-moderate asthma who were followed prospectively for 6 months. All patients used an MDI equipped with an electronic doser attached to their inhaled steroid. At each 2-month follow-up visit, doser and canister weight data were recorded, while child and mother were interviewed separately regarding medication use. They concluded that children and mothers reported, on average, over 80% adherence with the prescribed inhaled steroid. However, canister weight revealed, on average, adherence of 69%, significantly lower than self-report. When adherence recorded by the electronic doser, average adherence was 50%.

Non-Compliance in COPD

Adherence patterns in COPD do not appear to be different than the details shown above for asthma. Cecere et al 2012 stated that “Adherence to long-acting inhaled medications among patients with COPD is poor.” A second study conducted by Bender et al 2006 used pharmacy records for their analysis. Bender et al stated “This pharmacy database study portrays medication adherence levels to be considerably lower than those reported in most clinical trials, suggesting that most adults taking FCS [fluticasone propionate/salmeterol combination] obtain a single fill before abandoning their controller medication.” A similar set of observations were reported by Lareau and Yawn, 2010: “The rate of >50% poor adherence in the COPD population is not surprising, because people with COPD usually have multiple morbidities and take an average of six medications.” Lareau and Yawn point out that: “Acute and maintenance treatment of COPD relies on inhaled agents to manage and control symptoms and/or complications of the disease, and prevent exacerbations.” In general, it appears that COPD patients take less than 50% of the medication that is need to control their symptoms and a great deal of this non-compliance occurs with inhaled medications.

Non-Compliance in Cystic Fibrosis

The literature on compliance in Cystic Fibrosis is considerably thinner than for asthma or COPD, likely reflecting the relatively small number of patients involved. Marciel et al, 2010 state: “Treatment regimens for patients with cystic fibrosis (CF) are time-consuming and complex, resulting in consistently low adherence rates.”

Cost of Non-Compliance

The costs of non-compliance are two-fold: the patient experiences a reduced quality of life and pressure on healthcare systems increases since non-compliant patients may experience worsening of their condition, requiring more costly acute medical interventions. Stern et al. (2006) examined the association between medication compliance and exacerbation in asthmatics patients. This study showed that more compliant patients were significantly less likely to experience an asthma exacerbation than less compliant patients were.

LABAs and Steroids have Safety Issues

Last and of considerable important, LABAs are associated with a higher risk of death and steroids can cause growth retardation and bone loss (Walters, et al 2007), (Allen, 2005). Furthermore, inhaled drugs have been associated with poor oral health such as dental caries, candidiasis, ulceration, gingivitis, periodontitis, halitosis and taste changes (Godara, et al 2011).

Oral Dosage Forms Improve Compliance and Health Outcomes

Oral drug delivery would provide the benefit of improved compliance in these Orphan respiratory disorders. A recent article in the New England Journal of Medicine reported that oral drug compliance for respiratory diseases is 60% higher than comparable drugs delivered via the inhaled route (Price et al, 2011). Higher compliance is generally associated with improved health outcomes (Richter et al, 2003).

Need for Safer Oral Respiratory Treatments

The limitations of current inhaled drugs strongly suggests that an improved oral medication for Orphan Respiratory Disease is very much needed. An older drug that combines both bronchodilation and anti-inflammatory effects in one oral drug is theophylline. However, theophylline has serious cardiac, CNS and gastric side effects and is a narrow therapeutic index drug. These side effects severely limit the usefulness of theophylline in the treatment of Orphan Diseases or any disease. The use of theophylline is complicated by its serious, life threatening interactions with various drugs and that it has a narrow therapeutic index, so its use must be monitored to avoid toxicity. It can also cause nausea, diarrhea, increase in heart rate, arrhythmias, CNS excitation (headaches, insomnia, irritability, dizziness and lightheadedness) and death. Its toxicity is increased by erythromycin, cimetidine, and fluoroquinolones, such as ciprofloxacin (package insert, Theo-24). Ten percent of patients treated in an emergency setting for theophylline overdose die (Paloucek et al, 1988). Seizures can also occur in severe cases of toxicity and is considered to be a neurological emergency (Yoshikawa, 2007). Theophylline is a member of a class of drugs known as xanthines and drugs such as caffeine, aminophylline and bromophylline all produce elevated heart rates, CNS simulation and gastric upset.

Significant Need for a Safer Xanthine Class Drugs for Orphan Respiratory Diseases

Doxofylline is a xanthine but has been shown to have a superior side effect profile as compared to theophylline with fewer CNS, cardiac and gastric side effects (Page, 2010). In one large, randomized clinical study comparing doxofylline to theophylline, 31% of the theophylline subjects dropped out of the study due to side effects but only 11% dropped out of the doxofylline arm due to side effects (Goldstein et al 2002). In this study, doxofylline was as effective as theophylline in treating chronic asthma.

The use of doxofylline is designed to combine two key treatment results, bronchodilation and anti-inflammatory effects in one composition with a superior side effect and safety profile as compared to either theophylline or the combination of a LABA and a steroid.

Surprisingly and unexpectedly, the use of doxofylline to treat these Orphan Diseases has never been revealed in any publication, patent or patent application.

### Chemical Structure and Formula for Doxofylline

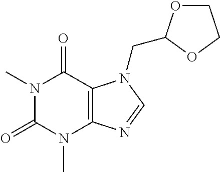 [SMILES: Cn1c(=O)c2c(ncn2CC2OCCO2)n(C)c1=O]

### 7-(1,3-dioxolan-2-ylmethyl)-1,3-dimethylpurine-2,6-dione

### Summary of Orphan Disease Treatment.

As noted earlier, bronchodilators and anti-inflammatory drugs have been used together in the treatment of the respiratory Orphan Diseases under consideration here. However, only xanthine class drugs offer both effects in one drug. An improved xanthine product with a better safety profile could be a valuable addition to treatment of these Orphan Diseases. Doxofylline is a xanthine with an improved side effect profile with a similar level of efficacy as compared to theophylline in the treatment of asthma (Goldstein et al, 2002). As previously noted, the most striking difference relative to the side effects reported by Goldstein over the 12 week testing period was the difference in dropout rate during the study. Subjects discontinuing treatment (drop outs) is an important, objective measure of the relative toxicity of one drug as compared to a second. Importantly, doxofylline and theophylline had very similar levels of efficacy in these asthma patients.

Equally important, doxophylline can be delivered orally versus the use of inhaled delivery for long acting beta agonists and steroids. Oral delivery would yield the benefit of improved compliance and higher compliance is generally associated with improved health outcomes.

It is the object of the present invention to utilize an oral dosage form containing doxofylline to treat the Orphan Respiratory Diseases noted above to improve long function via the bronchodilation and anti-inflammatory effects of doxofylline. Such a use of doxofylline has never been revealed in the published literature or issued or pending patents.

The history of prior art for the treatment of these Orphan Respiratory Diseases indicates that a serious need exists for a novel and useful treatment that that provides an unexpected advancement in the science Orphan Respiratory Disease management. For example, the prior art does not provide for xanthine based drugs that have a tolerable side effect profile while delivering the twin benefits of bronchodilation and anti-inflammatory effects to treat Orphan Respiratory Disease patients. Present treatments must combine a bronchodilator and an anti-inflammatory drug to achieve both effects. Further, long acting beta agonists (LABAs) and inhaled steroids both have deleterious health effects associated with them (Walters et al, 2007), (Allen, 2005). Theophylline combines both bronchodilation and anti-inflammatory properties in one oral drug but theophylline is a narrow therapeutic index drug that has serious side effects (Theo-24 Package Insert, Paloucek et al, 1988). These very serious, life threatening side effects and the narrow therapeutic window fundamentally limit theophylline's utility in the treatment of these Orphan Respiratory Diseases.

Accordingly, the present invention provides both novel and useful treatment effects for Orphan Respiratory Disease patients with an oral xanthine class drug that has a superior side effect profile as compared to theophylline. Doxofylline provides the dual benefits of bronchodilation and anti-inflammatory effects in an oral dosage form without the serious side effects of theophylline. Doxofylline can also overcome the safety and compliance problems associated with delivering a bronchodilator such as a long acting beta agonist or an anti-inflammatory drug such as a steroid via the inhaled route.

### SUMMARY OF THE INVENTION

The invention is a method for treating a human respiratory disease, said method comprising administering an effective amount of a pharmaceutical composition comprising doxofylline in an oral formulation to a patient having a condition selected from the group consisting of cystic fibrosis, brochiolitis obliterans, bronchiectasis, emphysema/COPD secondary to antitrypsin deficiency, reactive airway dysfunction syndrome in 9/11 First Responders and Survivors, World Trade Center Cough in 9/11 First Responders and Survivors, and orphan obstructive lung disease.

### BRIEF DESCRIPTION OF THE DRAWINGS

FIG. 1 is a flow chart that illustrates a process for creating an immediate release formulation in a hard gelatin capsule.

FIG. 2 is a flow chart that illustrates a process for creating an immediate release formulation in a compressed tablet.

FIG. 3 is a flow chart that illustrates a process for creating a sustained release formulation in a compressed tablet 3.

### DETAILED DESCRIPTION OF THE INVENTION

The invention is a method for treating a human respiratory disease, said method comprising administering an effective amount of a pharmaceutical composition comprising doxofylline in an oral formulation to a patient having a condition selected from the group consisting of cystic fibrosis, brochiolitis obliterans, bronchiectasis, emphysema/COPD secondary to antitrypsin deficiency, reactive airway dysfunction syndrome in 9/11 First Responders and Survivors, World Trade Center Cough in 9/11 First Responders and Survivors, and orphan obstructive lung disease,

Doxofylline combines two highly desirable treatments for Orphan Respiratory Diseases (ORD), bronchodilation and anti-inflammatory effects in one oral drug. Previously, only theophylline was available to treat Orphan Respiratory Diseases but theophylline has many very serious side effects that limit its utility. The present method of treating patients having such Orphan diseases with doxofylline is characterized as safer to use by patients because doxofylline is not a narrow therapeutic index drug like theophylline. Furthermore, doxofylline does not have the serious drug interactions that limit the use of theophylline with many other compounds such as cimetidine or fluoroquinolones. Additionally, doxofylline is safer than the Long Acting Beta Agonists (LABA) for the treatment of Orphan Respiratory Diseases because LABAs are known to cause death in some patients. Furthermore, doxofylline does not cause growth retardation in children as does the use of steroids to treat Orphan Respiratory Diseases. Steroids are also known to cause bone loss in adult patients whereas doxofylline does not.

The method of treating ORDs with an oral formulation of doxofylline will lead to greater compliance and persistence than inhaled drugs used to treat ORDs. Greater compliance will lead to better health outcomes as compared to treatment methods that result in lower compliance such as the use of inhaled drugs to treat ORDs.

The method of treating ORD patients with doxofylline can utilize pharmaceutical compositions formulated, for example, as a capsule or compressed tablet or as a liquid or a suspension. Furthermore, the method of the invention may utilize compositions which would deliver either an immediate release effect (IR) or a sustained release effect (SR) or both an IR and SR effect. This will involve the use of an IR component and sustained release (SR) component in the pharmaceutical composition.

In addition, the method of the invention may utilize doxofylline combined with an additional medicinal component that would constitute a fixed dose combination product. Doxofylline may also be used concomitantly with additional medicinal agents that are useful to treating ORDs.

By “9/11 First Responder” is meant public safety personnel such as fireman, policemen, Emergency Medical Technicians (EMT), nurses, doctors, and other personnel who responded on scene to the World Trade Center attack on Sep. 11, 2001 and the subsequent time frame covering the debris removal and search for survivors and remains at the site. Survivors are those who worked at the World Trade Center or lived in the immediate environs. Such persons are known to be listed in the World Trade Center Health Registry as defined at http://www.nyc.gov/html/doh/wtc/html/registry/about.shtml.

By “orphan respiratory disease” is meant any disease primarily affecting the function of the nose, larynx, trachea, lungs, bronchi, alveoli and associated muscles in the respiratory system which facilitate the oxygenation of the blood with a concomitant removal of carbon dioxide and other gaseous metabolic wastes from blood and the disease afflicts less than 200,000 people in the United States.

The term “obstructive lung disease” means any disease that causes the airways of the lungs to become narrow or blocked so that a patient cannot exhale completely. Because of damage to the lungs or narrowing of the airways inside the lungs, exhaled air comes out more slowly than normal. At the end of a full exhalation, an abnormally high amount of air may still remain in the lungs.

By “orphan obstructive lung disease” is meant any orphan respiratory disease that includes obstructive lung disease.

By “pharmaceutically active agent” is meant agents other than food articles that are intended to diagnose, cure, mitigate, treat or prevent disease in man or other animals or that are intended to affect the structure or any function of the body of man or other animals that are physiologically acceptable. The agent could be a combination of drug therapies as well as a single agent.

By “physiologically acceptable” is meant those substances that are adequately tolerated without causing unacceptable negative side effects.

By “immediate release”/IR is meant that the pharmacologically active agent is released from the formulation immediately such that at least 80%, preferably at least 85%, more preferably at least 90%, or most preferably at least 95% of the agent in the formulation is absorbed into the blood stream of a patient two hours after administration. Whether a pharmaceutical composition is formulated for immediate release can be determined by measuring the pharmacokinetic profile of the formulation.

By “extended release” is meant that the pharmaceutically active agent is released from the formulation at a controlled rate such that the formulation allows for a reduction in dosing frequency as compared to that presented by a conventional dosage form, e.g. an immediate release dosage form.

The invention also includes methods and compositions for delivering combinations of pharmaceutically active compounds. Examples of such combinations are:

A: doxofylline and a steroid

B: doxofylline and a long acting beta agonist

C: doxofylline and an anti-cholinergic agent

D: doxofylline and an antibiotic

E: doxofylline and a leukotriene inhibitor

F: doxofylline and an expectorant

### Dosage Forms

Suitable dosage forms include syrups, suspensions, tablets, capsules, granules, powders, pellets and the like.

### Solid Oral Dosage Delivery Systems

Suitable delivery systems include compressed tablets, capsules, granules/multi-particle systems, pellets as well as granules/multi-particle systems, pellets filled into capsules and the like

### Liquid Oral Dosage Delivery Systems

Suitable delivery systems include solutions and suspensions and the like.

### EXAMPLES

Each of the compositions of the examples below is useful for oral administration doxofylline.

### Example 1

IR dosage form, hard gelatin capsule

| | |
|---|---|
| Doxofylline | 400 mg |
| Microcrystalline cellulose | 200 mg |
| Modified Starch 1500 | 200 mg |
| Magnesium Stearate | 8 mg |
| Empty Capsule Shell #0 | 96 mg |
| Total Dosage Form Weight | 904 mg |

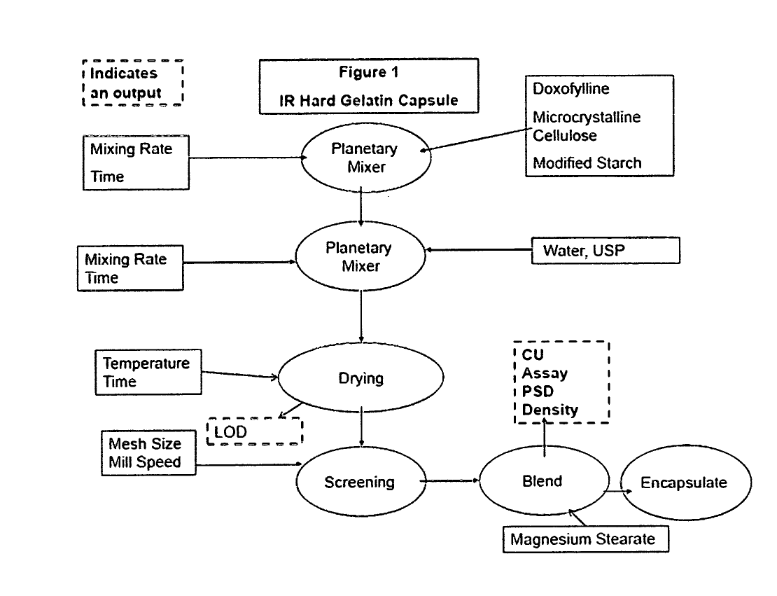

The immediate release capsules are manufactured using a standard wet granulation technique. The doxofylline, microcrystalline cellulose and modified starch 1500 are dry blended in a suitable mixer such as a planetary mixer. Water USP is then added to the dry powder blend while mixing until suitable granules are formed. The wet mass is dried to a level of approximately 1.5% loss on drying (LOD). The dried granules are then screened/milled to a suitable particle size, blended with the magnesium stearate and subsequently filled into hard gelatin capsules having a final filled weight of 904 mg. The dried blend may be tested for assay and content uniformity prior to the encapsulation. The process is shown in FIG. 1 below.

### Example 2

IR dosage form, compressed tablet

| | |
|---|---|
| Doxofylline | 400 mg |
| Microcrystalline cellulose | 200 mg |
| Polyplasdone XL | 20 mg |
| HPMC 3cps | 25 mg |
| Anhydrous Lactose | 200 mg |
| Magnesium stearate | 6 mg |
| Colloidal Silicon Dioxide | 6 mg |
| Total Dosage Form Weight | 857 mg |

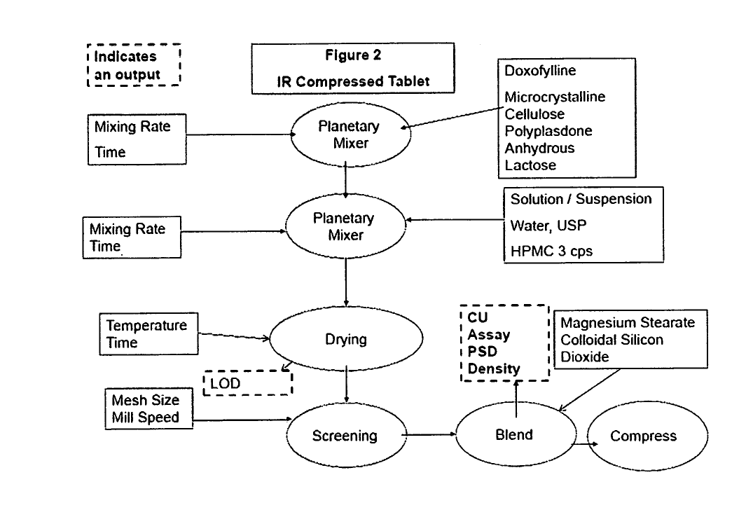

The immediate release tablets are manufactured using a standard wet granulation technique. The doxofylline, microcrystalline cellulose, Polyplasdone XL, and anhydrous lactose are dry blended in a suitable mixer such as a planetary mixer. The HPMC 3 cps is added to a sufficient amount of water USP to form a suspension/solution. This is then added to the dry powder blend while mixing until suitable granules are formed. The wet mass is dried to a level of approximately 1.5% loss on drying (LOD). The dried granules are then screened/milled to a suitable particle size, blended with the magnesium stearate and colloidal silicon dioxide and subsequently compressed into tablets having a final weight of 857 mg. The dried blend may be tested for assay and content uniformity prior to the compression. The process is shown in FIG. 2 below.

### Example 3

SR dosage form, compressed tablet

| | |
|---|---|
| Doxofylline | 600 mg |
| Microcrystalline cellulose | 120 mg |
| HPMC 100,000CPS | 185 mg |
| Anhydrous lactose | 170 mg |
| Magnesium stearate | 13 mg |
| Total Dosage Form Weight | 1,088 mg |

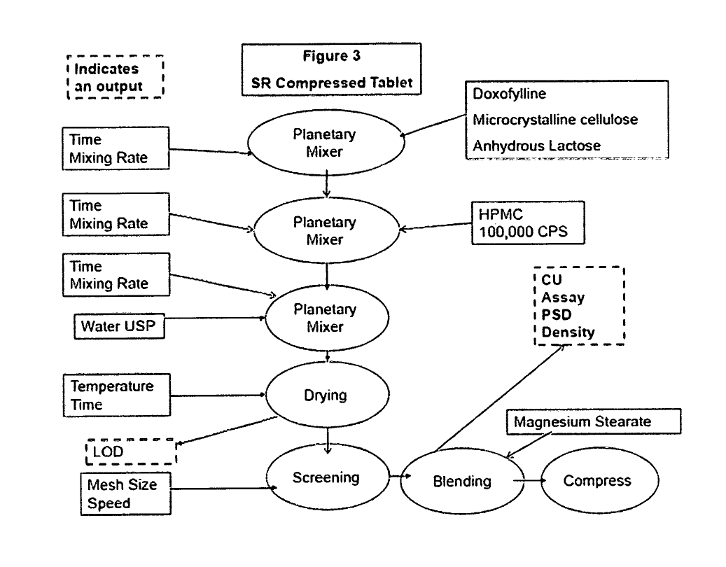

The sustained release tablets are manufactured using a two phase wet granulation technique. The doxofylline, microcrystalline cellulose and anhydrous lactose are dry blended in a suitable mixer such as a planetary mixer. The HPMC 100,000 cps is added to the blended powders and mixed. A sufficient amount of water USP is then added to the dry powder blend while mixing until suitable granules are formed. The wet mass is dried to a level of approximately 1.5% loss on drying (LOD). The dried granules are then screened/milled to a suitable particle size, blended with the magnesium stearate and subsequently compressed into tablets having a final weight of 1088 mg. The dried blend may be tested for assay and content uniformity prior to the compression. The process is shown in FIG. 3 below.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

### REFERENCES

### Articles and Books

Allen D B. Inhaled steroids for children: effects on growth, bone, and adrenal function. Endorinol Metab Clin North Am. 2005; 34(3): 555-64.
Balfour-Lynn I M, Welch K. Inhaled corticosteroids for cystic fibrosis. Cochrane Database Syst Rev. 2009 Jan. 2; (1): CD001915.
Bender B, Wamboldt F, O'Connor S, Rand C, Szefler S, Milgrom H and Wamboldt M. Measurement of children's asthma medication adherence by self report, mother report, canister weight and doser ct. Ann Allergy Asthma Immunol, 2000, 85(5): 416-21.
Bender B, Pedan A, Varasteh, L. Adherence and persistence with fluticasone propionate/salmeterol combination therapy. J Allergy Clin Immunol, 2006, 118(4):899-904.
Bergeron A, Bell A, Chevret S, Ribaud P, Devergie A, Esperou H, Ades L, Gluckman E, Socie G and Tazi A. Combined inhaled steroids and bronchodilators in obstructive airway disease after allogenic stem cell transplantation. Bone Marrow Transplantation, 2007, 39, 547-533.
Brooks S M, Weiss M A and Bernstein I L. Reactive airways dysfunction syndrome (RADS): persistent asthma syndrome after high level irritant exposures. Chest, Volume 88, 1985, 376-384.
Cecere Laura M. Cecere, Christopher G. Slatore, Jane E. Uman, Laura E. Evans, Edmunds M. Udris, Chris L. Bryson, and David H. Au Adherence to Long-Acting Inhaled Therapies among Patients with Chronic Obstructive Pulmonary Disease (COPD). 251 COPD, 9:251-258, 2012
Chatkin J, Cavalet-Blanco D, Scaglia N, Tonietto R, Wagner M, Fritscher C. Compliance with maintenance treatment of asthma. J Brass Pneumol. 2006; 32(4):277-83.
Colby, T. V. Bronchiolitis, Pathologic Consideration. Am J Clin Pathology; 109:101-9, 1998.
Corda L, Bertella E, La Pian G E, Boni E, Redolfi S and Tantucci C. Inhaled corticosteroids as additional treatment in alpha-1-antitrypsin-deficiency-related COPD. Respirations, 2008; 76; 61-68.
Colombo C, Russo M C, Zazzeron L, Romano. Liver disease in cystic fibrosis. J. Pediatr. Gastroenterol. Nutr. 43 Suppl 1: S49-55.
Drug Topics, 2010 Top 200 Branded Drugs by Total Prescriptions accessed at http://drugtopics.modernmedicine.com/drugtopics/data/articlestandard//drugtopics/252011/72725 6/article.pdf, Sep. 9, 2012.
Epler, G R. Diagnosis and treatment of constrictive bronchiolitis. F1000 Medicine Reports, 2010, 2:32 (doi:10.3410/M2-32).
Feldman C., Bronchiectasis: New Approaches to Diagnosis and Management; Clin Chest Med 32, 535-546, 2011.
Frankel S K, Schwarz M I. Update in idiopathic pulmonary fibrosis. Curr Opin Pulm Med.; 15(5):463-9, 2009.
Friedman, S, Cone J, Eros-Samyai M, Prezant D, Szeinuk J, Clark N, Milek D, Levin S, Gillor. Clinical guidelines for adults exposed to World Trade Center Disaster (Respiratory and Mental Health). City Health Information, NYC Department of Health and Mental Hygiene, September 2006.
Godara N, Godara R, Khullar M. Imapct of inhalation therapy on oral health. Lung India, 2011; 28(4); 272-5.
Goldstein M F and Chervinsky, P. Efficacy and safety of doxofylline compared to theophylline in chronic reversible asthma—a double-blind randomized placebo-controlled multicenter clinical trial. Med Sci Monit, 2002; 8(4): CR297-304
Hardin D S (August 2004). GH improves growth and clinical status in children with cystic fibrosis—a review of published studies. Eur. J. Endocrinol. 151 Suppl 1: S81-5.
Kaplan A and Cosentino L. α-Antritrpsin deficiency. Canadian Family Physician, 2010; 56:19-24.
Lacasse Y, Archibald H, Ernst P, Boulet L. Patterns and determinants of compliance with inhaled steroids in adults with asthma. Can Respir J. 2005, 12(4):211-7.
Lareau S and Yawn B. Improving adherence with inhaler therapy in COPD. International Journal of COPD, 2010, 5, 401-406.
Makker K, Agarwal A, Sharma R. Oxidative stress & male infertility. Indian J. Med. Res. 129 (4): 357-67, 2009.
Marciel K, Saiman L, Quittell L, Kawkins M and Quittner A. Cell phone intervention to improve adherence. Pediatr Pulmonol, 2010, 45(2): 157-164.
Martinez-Garcia M, Soler-Cataluna J, Catalan-Serra, P, Roman-Sanchez, P and Tordera M P. Clinical Efficacy and Safety of Budesonide-Formoterol in Non-Cystic Fibrosis Bronchiectasis. Chest, 141, 461-468, 2011.
Mitchell, Richard Sheppard; Kumar, Vinay; Robbins, Stanley L.; Abbas, Abul K.; Fausto, Nelson (2007). Robbins Basic Pathology, Saunders/Elsevier. ISBN 1-4160-2973-7.
Needham M, Stockley R A. α1-Antitrypsin deficiency•3: Clinical manifestations and natural history. Thorax 59 (5): 441-5, 2004.
O'Donnell A E. Bronchiectasis. Chest; 134:815-823, 2008.
O'Malley C A. Infection control in cystic fibrosis: cohorting, cross-contamination, and the respiratory therapist. Respir Care 54 (5): 641-57, 2009
Page P. Doxofylline: A “Novofylline”. Pulmonary Pharmacology & Therapeutics, 2010, 23:231-234.
Pan S H, Canafax D M, Le C T, Cipolle R J, Uden D L and Warwick W J. Bronchodilation from intravenous theophylline in Patients with cystic fibrosis: Results of a blinded placebo-controlled crossover clinical trial. Pedatric Pulmonol. 1989; 6:172-179.
Paloucek F P, Rodvold, K A. Evaluation of theophylline overdoses and toxicities. Annal of Emergency Medicine, 1988; 17: 135-144.
Price D, Musgrave S D, Shepstone L, Hillyer E V, Sims E J, Gilbert R F T, Juniper E F, Ayres J G, Kemp L, Blyth A, Edward M A, F. Wilson, M., Wolfe S, Freeman D, Mugford M, Murdoch J, and Harvey I. N Engl J Med 2011; 364:1695-1707.
Richter A, Anton S, Koch P, and Dennett S. The Impact of Reducing Dose Frequency on Health Outcomes. Clinical Therapeutics (2003); 25(8):23072335.
Stern L, Berman J, Lumry W, Katz L, Want L, Rosenblast L, Doyle J. Medication compliance and disease exacerbation in patients with asthma: a retrospective study of managed care data. Annals of Allergy, Asthma & Immunology. 97(3): 402-408.
Stoller J, Aboussouan L. Alpha1-antitrypsin deficiency. Lancet 365 (9478): 2225-36, 2005.
Sullivan P, Bekir S, Jaffar Z, Page C P, Jeffery P, Costello J. Anti-inflammatory effects of low-dose oral theophylline in atopic asthma. Lancet 1994; 343:1006e8.
Theo-24, package insert. Available at U. S, National Library of Medicine website at http://dailymed.nlm.nih.gov/dailymed
Tsang K W, Tan K C and Ho P L. Inhaled fluticasone in bronchiectasis: a 12 month study. Thorax, 60:239-243, 2005.
Walters E H, Gibson P G, Laserson, T J, Walters J A. Long-acting beta2-agonists for chronic asthma in adults and children where background therapy contains varied or no inhaled corticosteroid. Cochrane Database Syst Rev., 2007 Jan. 24; (1): CD0011385.
Wedzicha J, Decramer M, Seemungal T. The role of bronchodilatory treatment in the prevention of exacerbations of COPD. European Respiratory Journal, 2012 (Epub ahead of print).
Weiden M, Errier N, Nolan A, Rom W, Comfort A, Gustave J, Zeirg-Owens R, Zheng S, Goldring R, Berger K, Coxenza K, Lee R, Webber M, Kelly K, Aldrich T Prezant D. Obstructive airways disease with air trapping among firefighters exposed to World Trade Center dust. Chest, 2010; 137(3):566-574.
Yankaskas J R, Marshall B C, Sufian B, Simon R H, Rodman D. (2004). Cystic fibrosis adult care consensus conference report. Chest, 2004 January: 125(1 Suppl): 1S-39S.
Yoshikawa H. First-line therapy for theophylline-associated seizures. Acta Neurol Scand 115 (4 Suppl): 57-61, 2007.

### Patents

None

What is claimed is:

1. A method for providing improved compliance and safety with a treatment regimen for a patient having a human respiratory disease, selected from the group consisting of cystic fibrosis, bronchiolitis obliterans, and bronchiectasis, said method and treatment regimen consisting of administering to said patient a pharmaceutically active agent consisting of doxofylline in an oral dosage form.

2. The method of claim 1 wherein said oral dosage form is comprised of doxofylline in an amount from about 50 mg to about 2,000 mg.

3. The method of claim 1, wherein said oral dosage form is formulated as a member of the group consisting of a capsule, a powder, a thin film, a caplet and a tablet.

4. The method of claim 1, wherein said oral dosage form is formulated as a solution.

5. The method of claim 1, wherein said oral dosage form is formulated as a suspension.

6. The method of claim 1, wherein the respiratory disease being treated is cystic fibrosis.

7. The method of claim 1, wherein the respiratory disease being treated is bronchiolitis obliterans.

8. The method of claim 1, wherein the respiratory disease being treated is bronchiectasis.

9. The method of claim 1, wherein the respiratory disease being treated is emphysema/COPD secondary to antitrypsin deficiency.

10. The method of claim 1, wherein the respiratory disease being treated is reactive airway dysfunction syndrome in 9/11 First Responders and Survivors.

11. The method of claim 1, wherein the respiratory disease being treated is World Trade Center Cough in 9/11 First Responders and Survivors

12. The method of claim 1, wherein the respiratory disease being treated is an orphan obstructive lung disease.
